SEQUENCE LISTING (1 sequence)

SEQ ID NO: 1 - DNA, 240 bp, Unknown
  Store Bought Purdue Chicken
gagctcacggggacagccccccgccaaagcccccagggatgtaattgcatccctcttccgctagggggcagcagcgagccgcccggggctccgctccggtccggcgcttcccccgcatccccgcgagccgagccggcagcgtgcggggacagcccggcacggggaaggtggcacgcgatcgtttcctctgaacgcttctcgctgctctttgagcctgcagacacctggggggatacgggg

(19) United States
(12) Patent Application Publication
(10) Pub. No.: US 2004/0002158 A1
(43) Pub. Date: Jan. 1, 2004

(54) Long term expression of lentiviral vectors

(76) Inventor: Lung-Ji Chang, Gainesville, FL (US)

Correspondence Address: Stanley A. Kim, Ph.D., Esq., Akerman Senterfitt, Suite 400, 222 Lakeview Avenue, West Palm Beach, FL, 33402-3188, US

(21) Appl. No.: 10/455,080

(22) Filed: Jun. 5, 2003

Related U.S. Application Data
(60) Provisional application No. 60/385,986, filed on Jun. 5, 2002.
(60) Provisional application No. 60/385,864, filed on Jun. 5, 2002.

(51) Int. Cl. C12N 15/867; C12N 7/00; C07H 21/02; C12N 5/06

(52) U.S. Cl. 435/456; 435/320.1; 435/235.1; 435/325; 536/23.72

(57) ABSTRACT

Lentiviral vectors that include a transgene and one or more copies of the cHS4 insulator in a forward or reverse orientation, when expressed in cells, exhibit prolonged transgene expression compared to vectors lacking the insulator.

### CROSS REFERENCE TO RELATED APPLICATION

The present application claims the benefit of U.S. provisional patent application No. 60/385,986 filed Jun. 5, 2002.

### STATEMENT AS TO FEDERALLY SPONSORED RESEARCH

This invention was made with U.S. government support under grant numbers 2906420-12 and P50 HL59412 both awarded by the National Institutes of Health. The U.S. government may have certain rights in the invention.

### FIELD OF THE INVENTION

The invention relates to the fields of molecular biology, gene therapy, microbiology and virology. More particularly, the invention relates to compositions and methods for enhancing the long term expression of lentiviral vectors in cells.

### BACKGROUND

Long term transgene expression is required in gene therapy applications involving correction of genetic disorders and permanent phenotype introduction. Retroviral vectors have been the preferred tool for long term gene transfer because retroviruses employ a unique proviral chromosomal integration mechanism. However, ectopic retroviral gene transfer into host cell does not always ensure long lasting transgene expression. A heterologous promoter inserted in the murine leukemia oncoretroviral vector (MLV) may be silenced at different time following infection depending on the cell type. In embryonic carcinoma (EC), embryonic stem cells (ES) and early embryos, it has been shown that the suppression of MLV transgene expression occurs soon after infection through mechanisms involving loss of unintegrated proviral DNA and transcriptional silencing. The retroviral transgene silencing is controlled through negative cellular factors binding to the repressor binding sites in the long terminal repeats (LTRs) and the primer binding site (PBS) of the provirus involving DNA methylation and methylation-independent mechanisms.

Unlike the silencing observed using oncoretroviral vectors, several previous reports indicated that silencing was not observed using lentiviral vectors. In contrast, in the experiments described herein, silencing was observed using lentiviral vectors. The identification of this effect using lentiviral vectors indicates that there is a need to develop silencing-resistant lentiviral vectors for applications in which long-term transgene expression is desired, e.g., gene therapy applications.

### SUMMARY

The invention relates to the development of a new method for promoting the long-term expression of a transgene introduced into a cell using a lentiviral vector. This method utilizes regulatory elements known as “insulators” to prevent the silencing effect by shielding the transgene promoter from the influence of neighboring regulatory elements.

The invention also relates to lentiviral constructs that incorporate an insulator as well as a transgene. A particularly preferred insulator for use in the invention is the chicken β-globin HS4 insulator known as cHS4 (or simply HS4). This insulator was incorporated into lentiviral vectors and evaluated using two different cell types: TE671 (human rhabdomyosarcoma) and P 19 (embryonic stem cell). Increasing copy number of HS4 cloned in the viral LTR appeared to moderately interfere with the virus production. Without the insulator, a silencing effect in lentiviral transgene expression was observed in transduced TE671 cells after 15 passages, and the same effect was observed in P19 cells only after 2 passages. Lentiviral vectors with the HS4 insertion in either orientation, however, displayed significantly protection of transgene expression in both types of cells.

Accordingly, the invention features a nucleic acid molecule that include a first nucleotide sequence derived from a lentivirus, a second nucleotide sequence not derived from the lentivirus, and third nucleotide sequence that includes an insulator. The nucleic acid of the invention can be included in a plasmid. In other variations, an insulator is located upstream (5′ to) of the second nucleotide sequence.

Also within the invention is a cell into which a nucleic acid molecule of the invention has been introduced. The cell can be a stem cell such as an embryonic stem cell.

Another aspect of the invention is a method for promoting long term expression of a lentiviral vector in a cell. This method includes the step of introducing a nucleic acid molecule of the invention into the cell.

In the nucleic acid molecule, cell, and method of the invention, the insulator(s) can be a cHS4 insulator such as the cHS4 insulator that has the amino acid sequence of SEQ ID NO:1. The insulator(s) can be in a forward or reverse orientation.

Unless otherwise defined, all technical terms used herein have the same meaning as commonly understood by one of ordinary skill in the art to which this invention belongs. Commonly understood definitions of molecular biology terms can be found in Rieger et al., Glossary of Genetics: Classical and Molecular, 5th edition, Springer-Verlag: New York, 1991; and Lewin, Genes V, Oxford University Press: New York, 1994. Commonly understood definitions of virology terms can be found in Granoff and Webster, Encyclopedia of Virology, 2nd edition, Academic Press: San Diego, Calif., 1999; and Tidona and Darai, The Springer Index of Viruses, 1st edition, Springer-Verlag: New York, 2002. Commonly understood definitions of microbiology can be found in Singleton and Sainsbury, Dictionary of Microbiology and Molecular Biology, 3rd edition, John Wiley & Sons: New York, 2002.

Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control.

### BRIEF DESCRIPTION OF THE DRAWINGS

FIG. 1A is a series of highly schematic maps of plasmid constructs used for generating HIV-1 derived lentiviral vectors.

FIG. 1B is a nucleotide sequence of the cHS4 insulator used in the studies described below. The sequence of a variant of the cHS4 that was used in other studies is shown by the editorial markings below the main sequence. These include several substitutions, on deletion, and two insertions.

FIG. 1C is highly schematic overview of a making a proviral DNA from plasmid constructs according to the invention.

FIG. 2 is two graphs showing the results of a study on the expression of a nLacZ reporter gene on TE671 cells. A=2 MOI; B=5 MOI.

FIG. 3 is a graph showing the difference in decrease of expression of a transgene for different lentiviral constructs.

FIG. 4 is two graphs showing the results of a study on the expression of a nLacZ reporter gene on P19 cells. A=2 MOI; B=5 MOI.

FIG. 5 is a graph showing the effect of the use of an insulator on P19 cells.

### DETAILED DESCRIPTION

The invention provides methods and compositions for promoting the long-term expression of a transgene introduced into a cell using a lentiviral vector. In the embodiments described below, a number of lentiviral vectors based on human immunodeficiency virus type 1 (HIV-1) were constructed. These included one or more copies of a cHS4 insulator in a forward or reverse orientation. The long term expression of these HS4 lentiviral vectors was studied in two different cell types: TE671 (human rhabdomyosarcoma) and P19 (embryonic carcinoma cells). Increasing copy number of HS4 in the LTR appeared to moderately interfere with the virus production. Without the insulator, a silencing effect in lentiviral transgene expression was observed in transduced TE671 cells after 15 passages, and in P19 cells only after 2 passages. Lentiviral vectors with the HS4 insertion in either orientation, however, displayed significant protection of transgene expression in both cell types.

The below described preferred embodiments illustrate adaptations of these compositions and methods. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning, 3rd Edition, Sambrook and Russell, Cold Spring Harbor Press, 2001; and Current Protocols in Molecular Biology, ed. Ausubel et al., Greene Publishing and Wiley-Interscience, New York, 1992 (with periodic updates). Various techniques using polymerase chain reaction (PCR) are described, e.g., in Innis et al., PCR Protocols: A Guide to Methods and Applications, Academic Press: San Diego, 1990. PCR-primer pairs can be derived from known sequences by known techniques such as using computer programs intended for that purpose (e.g., Primer, Version 0.5, ©1991, Whitehead Institute for Biomedical Research, Cambridge, Mass.). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the present invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

### Lentiviral Vectors

A number of different lentiviral vectors are known including those based on naturally occurring lentiviruses such as HIV-1, HIV-2, simian immunodeficiency virus (SIV), feline immunodeficiency virus (FIV), bovine immunodeficiency virus (BIV) and others. See U.S. Pat. No. 6,207,455. Although the invention is described using HIV-1 based vectors, other vectors derived from other lentiviruses might also be used by adapting the information described herein. Because of the many advantages HIV-1 based vectors provide for gene therapy applications, these are presently preferred. See U.S. Pat. No. 6,531,123.

To render HIV-1 derived vectors safe and efficient for gene therapy applications, it is desirable to (1) delete a maximum amount of the virus sequence to avoid the production of wild type virus by recombination without interfering with the virus efficacy and (2) insert heterologous sequences to increase the efficacy of the vector. For example, because efficient synthesis of HIV-1 Gag-Pol requires tat activation of the LTR and the interaction of Rev-RRE to mediate nuclear export of mRNA, these functions should be retained. On the other hand, because the accessory gene functions of vif, vpr, vpu and nef have been shown to be dispensable for viral replication, one or more of these might be deleted.

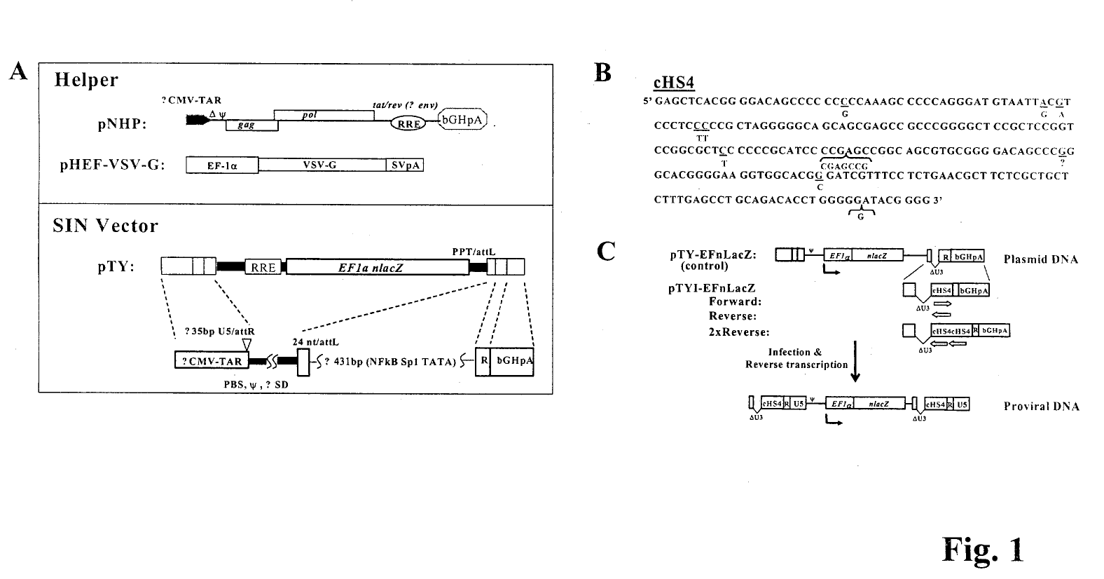

The lentiviral vectors of the invention might also be pseudotyped, e.g., to overcome restricted host cell tropism. For example, lentiviral vectors pseudotyped with vesicular stomatitis virus G (VSV-G) viral envelopes might be used. In addition, the potential risk of wild type recombination can be reduced by designing a three-plasmid co-transfection strategy for vector production. For example, referring to FIG. 1A, a three-plasmid design includes a helper construct, pNHP, that encodes the gag-pol (necessary viral proteins), a transducing vector construct, pTV, that encodes the viral genome which carries a foreign gene cassette (reporter gene), and a VSV-G envelope expression plasmid, pHEF-VSV-G. To increase vector titer in the system, an additional eukaryotic expression plasmid (e.g., a transactivator plasmid construct such as pCEP4-tat) might also be utilized.

To enhance safety, a self-inactivating (SIN) lentiviral vector might also be used. For example, a SIN lentiviral vector can be made by inactivating the 3′ U3 promoter and deleting of all the 3′ U3 sequence except the 5′ integration attachment site which is important for the integration into host chromosome. A particularly preferred construct for designing vectors of the invention is pTY shown in FIG. 1A.

### Insulators

Methods and compositions of the invention utilize insulators to promote long-term expression of a transgene in a cell by preventing the silencing effect caused by other regulatory elements. The insulator used in the embodiments described herein is a chicken HS4 insulator element (cHS4). The amino acid sequence of the particular cHS4 is provided herein as SEQ ID NO:1; although other versions of cHS4 that can serve as an insulator are known (see, e.g., Chung et al., Proc. Natl. Acad. Sci. USA, 94:575-580, 1997). In addition to cHS4, a number of other insulators are known. For a review, see Pannell et al., Rev. Med. Virol. 11:205-217, 2001. These might also be used in designing vectors for use in the invention. For example, the scs (scs sequence flanking the 87A1 hsp70 locus), BEAD-1, and gypsy (340 bp fragment from the gypsy retrotransposon) insulators might be used. See Modin et al., J. Virol. 74:11697-11707, 2000; Pamell et al., EMBO J. 19:5864-5874, 2000; and Biochem. Biophys. Res. Commun. 284:987-992, 2001.

### EXAMPLES

### Example 1

### Materials and Methods

As shown in FIG. 1C, three different clones were generated: (1) pTYcHS4-EFnLacZ forward, a construct that includes one copy of the cHS4 fragment of SEQ ID NO:1 in forward orientation; (2) pTYcHS4-EFnLacZ reverse, a construct that includes one copy of the cHS4 fragment of SEQ ID NO:1 in reverse orientation; and (3) pTYcHS4-EFnLacZ2xReverse, a construct that includes two copies of the cHS4 fragment of SEQ ID NO:1 in reverse orientation. After confirmation by DNA sequencing, these plasmids were retransformed and produced in a large scale and pure quality.

Cell Culture. TE671 cells were cultured in Dubelcco's modified Eagle's minimal essential medium (DMEM) supplemented with 10% heat inactivated (56° C., 30 minutes) fetal bovine serum (FBS, Gibco BRL) and 1% antibiotics penicillin/streptomycin, in a humidified atmosphere of 5% CO₂ in air at 37° C. P19 cells were cultured in minimal essential medium (MEM) supplemented with the same FBS and antibiotic as above. Cells were sub-cultured every two-three days (when confluent enough) by trypsinization.

DNA Transfection. Viruses were generated by co-transfecting 293T cells with five plasmids: pNHP, pTY, pHEF-VSV-G, pHEF-eGFP (as transfection control), and a tat plasmid. A modified DNA transfection protocol using the Superfect kit (Qiagen) was performed. For a six well plate, cells were split exactly 17 hours prior to transfection at about 90% confluency (9×10⁵ to 1×10⁶ cells per well). To be sure that the cells were split without clumps, they were trypsin treated at 37° C. for 5 minutes. The next morning, the media was removed and the cells were fed with 600 μl of fresh growth DMEM with 10% FBS. In an eppendorf tube were mixed: 75 μl (per well) of serum free DMEM; 2.7 μg of helper DNA mix (1 μg/μl) containing 1.8 μg of pNHP, 0.5 μg of pHEF-VSV-G, 0.2 μg of pCEP4tat, and 0.2 μg of pHEF-eGFP; and 0.8 μg of pTY DNA vector. After vortexing, 7 μl of Superfect (2:1 Superfect versus DNA) were added to the center of the tube, and mixed immediately by pipeting up and down five times. The mixture was then incubated at room temperature for 5 to 10 minutes. To the six well culture plate (with 600 μl of growth media) the DNA mix was added dropwise. The plate was then gently mixed by tilting back and forth a few times, and incubated at 37° C. in a humidified atmosphere of 3% CO₂ in air for 4-5 hours. After incubation, the media was removed, the cells were washed with the desired culture media and fed with 1.5 ml of culture media per well. Virus was collected in 12 hours periods for three times (24 h, 36 h and 48 h) and stored at −80° C. for further use.

Virus Transduction and Titration. Virus supernatants were filtered using a 0.45 μm low protein-binding filter to remove cell debris from transfected culture. The cells were split (TE671 or P19) at about 90% confluency and seeded in wells of a 24-well culture plate. The cells were incubated at 37° C. in a humidified atmosphere of 5% CO₂ for 2-4 hours or overnight. The media was then removed from the cells, and 200-300 μl/well (just enough to cover the cells) of media containing 8 μg of polybrene/ml of media were added. (DMEM for TE671 and MEM for P19).

For lentivirus titration, different volumes of virus stock were used, usually, 1, 5 and 10 μl for titer between 104 and 10. These volumes of virus stock were added to the media and mixed by swirling the plate. The cells were incubated at 37° C. in a humidified atmosphere of 5% CO₂ overnight. The next day, 0.5 ml of growth media was added directly to the infected culture without removing the old media. The cells were incubated in the same conditions as above for 24 hours (the incubation can be up to 48 hours from the time the virus was added).

The cells were then assayed for nuclear lacZ enzyme. First, the cells were washed twice with PBS and fixed for exactly 5 minutes at room temperature with 300 μl of fixative solution containing: 1% formaldehyde (0.27 ml of 37.6% for final 10 ml) and 0.2% glutaraldehyde (80 μl of 25% for final 10 ml) in PBS. The reaction was stopped by adding 500 μl of PBS in each well. The cells were then washed three times with PBS and incubated overnight with the conditions described before in 300 μl of staining solution containing: 4 mM K-Ferrocyanide, 4 mM K-Ferricyanide, 2 mM MgCl₂ and 0.4 mg/ml X-Gal in PBS. The next day, the number of blue cells were counted directly using an inverted microscope. The best titer was usually observed for the virus harvested 36 hours after transfection.

### Example 2

### Results

Lentiviral Vector Cosntruction. Lentiviral vectors carrying an internal human elongation factor-1α (EF1 α) promoter, a nuclear lacZ reporter gene, and cHS4 of SEQ ID NO:1 were constructed. The cHS4 fragment was inserted into the 3′ LTR of the lentiviral vector to generate three different constructs: pTYcHS4-EFnLacZ forward, pTYcHS4-EFnLacZ reverse, and pTYcHS4-EFnLacZ2xReverse (shown in FIG. 1C). During the reverse transcription, the cHS4 element was copied into the 5′ LTR of the vector. Thus, as shown in FIG. 1C, when integrated into the host chromosome, the reporter gene is flanked by the insulator.

As safety is a major concern with HIV-derived vectors, the pNHP/pTY vector system was developed to minimize the possibility of homologous recombination and replication competent virus (RCV) production. To examine vector efficacy, human 293T cells were co-transfected with the following five plasmids: pNHP; pHEF-VSV-G (an envelope expression plasmid); pTYSalIEFnLacZ, pTYcPPTEFnLacZ (controls), or one of the pTYcHS4 constructs shown in FIG. 1C (a transducing pTY construct); pCEP4tat (a tat plasmid); and pHEFeGFP (an internal transfection control). The vector titer was determined by titration on TE671 and P19 cells using transfected culture supernatants. The reporter gene LacZ was assayed by colorimetric staining for β-galactosidase activity. Results are shown in Table 1.

As reported in Table 1, every construct successfully produced viral vectors. The insulated vectors had titers close to the control (pTYSalIEFnLacZ), demonstrating that the insulator fragments had no adverse effect on the virus production. This was not true for the construct with two copies of the cHS4 fragment, probably because the fragment of about 500 bp affected the function of the LTR and therefore the production of virus. The pTYcPPTEFnLacZ is considered a control for this study because it does not contain any insulator fragment. However, it contains a cPPT sequence that has been cloned to increase the efficacy of the vector transduction.

TABLE 1
vector production in TE671, 36 h after transfection.
(tu: transducing unit)

| Vector | TE671 titer (tu/ml) | P19 titer (tu/ml) |
|---|---|---|
| pTYSaIIEFnLacZ | 4.22 × 10⁶ | 2.87 × 10⁵ |
| pTYcPPTEFnLacZ | 1.23 × 10⁷ | 2.1 × 10⁶ |
| pTYcHS4-EFnLacZ | 3.16 × 10⁶ | 3.27 × 10⁵ |
| forward | | |
| pTYcHS4-EFnLacZ | 3.4 × 10⁶ | 2.64 × 10⁵ |
| reverse | | |
| pTYcHS4EFnLacZ | 6.72 × 10⁵ | 8 × 10⁴ |
| 2 × reverse | | |

Analysis of Long-Term Expression in Cells. To investigate whether the cHS4 insulator can protect the transgene from the silencing effect, a long-term in vitro study was carried out. Two different cell types, TE671 and P19 cells were transduced with either the controls without the insulator or one of the three different constructs with the cHS4 element.

For each type of cell, two sets of transduction were carried out. For one set, cells were transduced at 2 MOI (multiplicity of infection), and for the other, they were transduced at 5 MOI in a total of two rounds of infection. Transduced cells were grown until confluent (4 days), trypsinized and plated into 6-well culture plates. Later, they were cultured into T-25 flasks to avoid contamination during handling. The transduced cells were continuously propagated without selection. At different passage times, some cells were frozen (for further experiments) and the percentage of nLac Z expressing cells was determined.

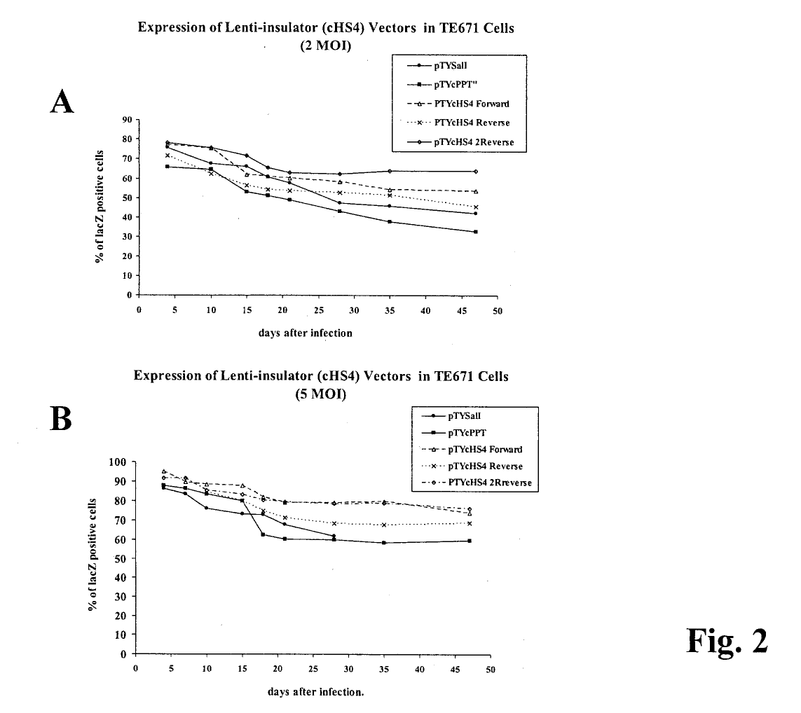

Referring to FIGS. 2A and 2B, using TE671 cells, the efficacy of transduction for all the constructs is about the same at the beginning of the study, 4 days after the first infection. At 2 MOI, the efficacy is between 66% for the pTYcPPTEFnLacZ and 78% for the pTYcHS4-EFnLacZ.

After 47 days of study, several things had been observed. First, for the study at both 2 and 5 MOI, all the vectors displayed decreased kinetics in the percentage of infected cells. The results are consistent between the 2 MOI and the 5 MOI experiments. The pTYcPPTEFnLacZ vector, which had the highest titer of infection (see Table 1), appeared to infect the least number of cells at the beginning of the study. The constructs with the highest decrease in expression of the reporter gene were the two controls without cHS4 modification. This decrease of expression is about 33% at 2 MOI and 28% at 5 MOI. This suggests that the silencing effect occurred in these cells.

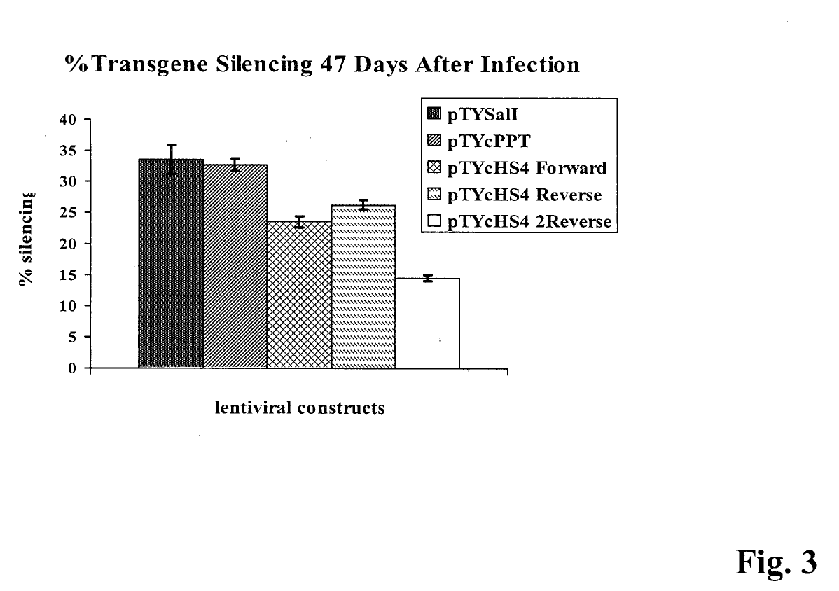

The decrease of expression of the transgene 47 days after infection is shown in FIG. 3. The construct with two copies of the cHS4 insulator appears to be the one that protects the expression of the reporter gene the best. The decrease of expression was only about 14.5% at 2 MOI and 15.6% at 5 MOI, which is half of what was observed for the two control constructs. It was also observed that the construct with a single copy of the cHS4 insulator in forward orientation seems to work better than the construct with a single copy of the cHS4 insulator in reverse orientation.

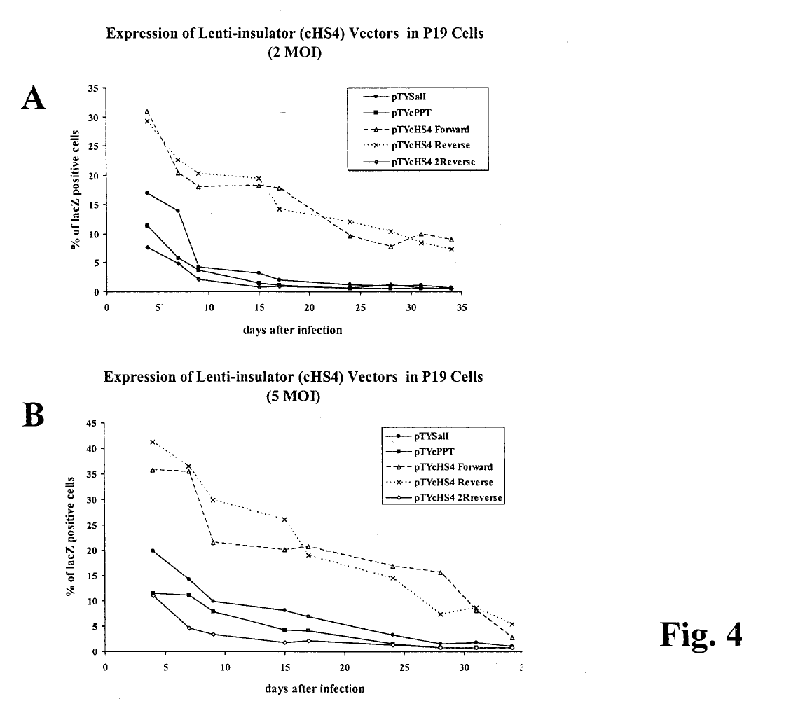

The same long-term study was carried out on P19 mouse embryonic stem cells. In previous studies, embryonic cells had been shown to have a silencing effect only three days after infection. This is probably due to their strong regulation system that allows them not to differentiate. Referring to FIGS. 4A and 4B, only four days after the first infection, the difference in the expression of insulated transgene and the control was already significant. A further experiment was performed to confirm these results. At day 0, the P19 cells were transduced at 5 MOI into a twelve well culture plate. At day 1, 24 hours after the first infection, a part of the cells was sampled for Lac Z assay and the percentage of cell transduced was determined. The result of this short-term study showed that all the constructs, including the control, transduced the P19 cells with the same efficiency.

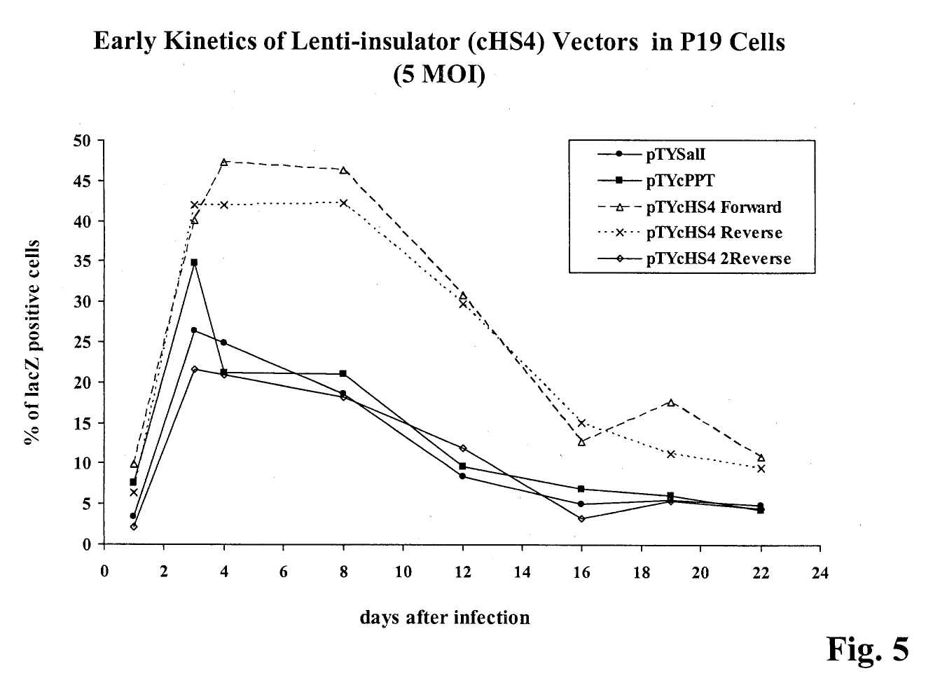

In an additional experiment, another portion of the cells was transduced a second time at 5 MOI and cultured. A second Lac Z assay was then performed 56 hours after the first infection. At this time point, the cells were transferred in a six well plate and LacZ assayed each time they were confluent (every three days). As shown in FIG. 5, after the second infection, a large number of cells were transduced and a difference between the insulated constructs and the control was observed. This demonstrated that the difference of transgene expression observed four days after infection was attributable to the activity of the insulator, rather than because more cells were transduced at the beginning. This also showed that the P19 cells were not transduced with the same efficacy as the TE671, even with two rounds of infection at 5 MOI. After the second infection, a higher percentage of infected cells was obtained for the pTYcHS4EFnLacZ forward with only 47% (when the lowest percentage observed in TE671 was at 2 MOI for the pTYcPPTEFnLacZ with 67% of transduced cells). The construct with two copies of the cHS4 in reverse orientation does not seem to insulate silencing (contrary to what was observed in the TE671 cells).

The gradual loss of transgene expression observed might have been due to transgene silencing or the loss of transduced cells. To distinguish between these two mechanisms, early and late passages of the same transduced cells were compared by Southern blot analysis using a control (pTYSalI) and an insulator vector (pTYcHS4Forward) to transduce both TE671 and P19. The genomic DNA was harvested and quantified and the same amount of DNA for each sample was used in the analysis. The results showed that within fifteen passages of TE671 cells, there was little to no loss of the integrated lentiviral transgene for both wild type and insulator vector transduced cells. However, P19 cells clearly demonstrated a rapid loss of lentiviral transgene after fifteen passages for cells transduced with either the wild type vector (pTYSalI) or the insulator vector (pTYcHS4Forward).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A nucleic acid molecule comprising a first nucleotide sequence derived from a lentivirus, a second nucleotide sequence not derived from the lentivirus, and a third nucleotide sequence that comprises an insulator.

2. The nucleic acid molecule of claim 1, wherein the insulator is a cHS4 insulator.

3. The nucleic acid molecule of claim 2, wherein the cHS4 insulator has the amino acid sequence of SEQ ID NO:1.

4. The nucleic acid molecule of claim 1, wherein the insulator is in a forward orientation.

5. The nucleic acid molecule of claim 1, wherein the insulator is in a reverse orientation.

6. The nucleic acid molecule of claim 1, further comprising a fourth nucleotide sequence that comprises an additional insulator.

7. The nucleic acid molecule of claim 1, wherein the third nucleotide sequence that comprises an insulator located upstream (5′ to) of the second nucleotide sequence.

8. The nucleic acid molecule of claim 1, wherein the nucleic acid molecule is comprised within a plasmid.

9. A cell into which has been introduced a nucleic acid molecule comprising a first nucleotide sequence derived from a lentivirus, a second nucleotide sequence not derived from the lentivirus, and a third nucleotide sequence that comprises an insulator.

10. The cell of claim 9, wherein the insulator is a cHS4 insulator.

11. The cell of claim 10, wherein the cHS4 insulator has the amino acid sequence of SEQ ID NO:1.

12. The cell of claim 9, wherein the cell is a stem cell.

13. The cell of claim 12, wherein the stem cell is an embryonic stem cell.

14. A method for promoting long term expression of a lentiviral vector in a cell, the method comprising the step of introducing into the cell a nucleic acid molecule comprising a first nucleotide sequence derived from a lentivirus, a second nucleotide sequence not derived from the lentivirus, and a third nucleotide sequence that comprises an insulator.

15. The method of claim 14, wherein the insulator is a cHS4 insulator.

16. The method of claim 15, wherein the cHS4 insulator has the amino acid sequence of SEQ ID NO: 1.

17. The method of claim 14, wherein the cell is a stem cell.

18. The method of claim 17, wherein the stem cell is an embryonic stem cell.
